SEQUENCE LISTING (10 sequences)

SEQ ID NO: 1 - DNA, 3031 bp, Homo sapiens
cctcgtgccgaattcggcacgaggcccgccggcggtggcgcggcggagacccggctggtataacaagaggattgcctgatccagccaagatgcagagcacttctaatcatctgtggcttttatctgatattttaggccaaggagctactgcaaatgtctttcgtggaagacataagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggagacaacaacaagacataaagtacttattatggaattttgtccatgtgggagtttatacactgttttagaagaaccttctaatgcctatggactaccagaatctgaattcttaattgttttgcgagatgtggtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttgcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtataaaataattacaggaaagccttctggtgcaatatctggagtacagaaagcagaaaatggaccaattgactggagtggagacatgcctgtttcttgcagtctttctcggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatattcatagctataatactgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctgaataccataggattaatatatgaaaaaatttccctccctaaagtacatccacgttatgatttagacggggatgctagcatggctaaggcaataacaggggttgtgtgttatgcctgcagaattgccagtaccttactgctttatcaggaattaatgcgaaaggggatacgatggctgattgaattaattaaagatgattacaatgaaactgttcacaaaaagacagaagttgtgatcacattggatttctgtatcagaaacattgaaaaaactgtgaaagtatatgaaaagttgatgaagatcaacctggaagcggcagagttaggtgaaatttcagacatacacaccaaattgttgagactttccagttctcagggaacaatagaaaccagtcttcaggatatcgacagcagattatctccaggtggatcactggcagacgcatgggcacatcaagaaggcactcatccgaaagacagaaatgtagaaaaactacaagtcctgttaaattgcatgacagagatttactatcagttcaaaaaagacaaagcagaacgtagattagcttataatgaagaacaaatccacaaatttgataagcaaaaactgtattaccatgccacaaaagctatgacgcactttacagatgaatgtgttaaaaagtatgaggcatttttgaataagtcagaagaatggataagaaagatgcttcatcttaggaaacagttattatcgctgactaatcagtgttttgatattgaagaagaagtatcaaaatatcaagaatatactaatgagttacaagaaactctgcctcagaaaatgtttacagcttccagtggaatcaaacataccatgaccccaatttatccaagttctaacacattagtagaaatgactcttggtatgaagaaattaaaggaagagatggaaggggtggttaaagaacttgctgaaaataaccacattttagaaaggtttggctctttaaccatggatggtggccttcgcaacgttgactgtctttagctttctaatagaagtttaagaaaagtttccgtttgcacaagaaaataacgcttgggcattaaatgaatgcctttatagatagtcacttgtttctacaattcagtatttgatgtggtcgtgtaaatatgtacaatattgtaaatacataaaaaatatacaaatttttggctgctgtgaagatgtaattttatcttttaacatttataattatatgaggaaatttgacctcagtgatcacgagaagaaagccatgaccgaccaatatgttgacatactgatcctctactctgagtggggctaaataagttattttctctgaccgcctactggaaatatttttaagtggaaccaaaataggcatccttacaaatcaggaagactgacttgacacgtttgtaaatggtagaacggtggctactgtgagtggggagcagaaccgcaccactgttatactgggataacaatttttttgagaaggataaagtggcattattttattttacaaggtgcccagatcccagttatccttgtatccatgtaatttcagatgaattattaagcaaacattttaaagtgaattcattattaaaaactattcatttttttcctttggccataaatgtgtaattgtcattaaaattctaaggtcatttcaactgttttaagctgtatatttctttaattctgcttactatttcatggaaaaaaataaatttctcaattttaatgtaaagaaaaaaaaaaaaaaaaaa

SEQ ID NO: 2 - protein, 729 aa, Homo sapiens
MQSTSNHLWLLSDILGQGATANVFRGRHKKTGDLFAIKVFNNISFLRPVDVQMREFEVLKKLNHKNIVKLFAIEEETTTRHKVLIMEFCPCGSLYTVLEEPSNAYGLPESEFLIVLRDVVGGMNHLRENGIVHRDIKPGNIMRVIGEDGQSVYKLTDFGAARELEDDEQFVSLYGTEEYLHPDMYERAVLRKDHQKKYGATVDLWSIGVTFYHAATGSLPFRPFEGPRRNKEVMYKIITGKPSGAISGVQKAENGPIDWSGDMPVSCSLSRGLQVLLTPVLANILEADQEKCWGFDQFFAETSDILHRMVIHVFSLQQMTAHKIYIHSYNTATIFHELVYKQTKIISSNQELIYEGRRLVLEPGRLAQHFPKTTEENPIFVVSREPLNTIGLIYEKISLPKVHPRYDLDGDASMAKAITGVVCYACRIASTLLLYQELMRKGIRWLIELIKDDYNETVHKKTEVVITLDFCIRNIEKTVKVYEKLMKINLEAAELGEISDIHTKLLRLSSSQGTIETSLQDIDSRLSPGGSLADAWAHQEGTHPKDRNVEKLQVLLNCMTEIYYQFKKDKAERRLAYNEEQIHKFDKQKLYYHATKAMTHFTDECVKKYEAFLNKSEEWIRKMLHLRKQLLSLTNQCFDIEEEVSKYQEYTNELQETLPQKMFTASSGIKHTMTPIYPSSNTLVEMTLGMKKLKEEMEGVVKELAENNHILERFGSLTMDGGLRNVDCL

SEQ ID NO: 3 - RNA, 19 nt, artificial
  oligonucleotide siTBK-1 sense
ggagacaacaacaagacau

SEQ ID NO: 4 - RNA, 20 nt, artificial
  oligonucleotide siTBK-1 antisense
augucuuguuguugucuccc

SEQ ID NO: 5 - DNA, 23 bp, artificial
  oligonucleotide TBK-1 sense
ttgaagaggagacaacaacaaga

SEQ ID NO: 6 - DNA, 19 bp, artificial
  oligonucleotide TBK-1 antisense
cattccacccaccacatct

SEQ ID NO: 7 - DNA, 20 bp, artificial
  oligonucleotide VEGF sense
cttgccttgctgctctacct

SEQ ID NO: 8 - DNA, 20 bp, artificial
  oligonucleotide VEGF antisense
gattctgccctcctccttct

SEQ ID NO: 9 - DNA, 20 bp, artificial
  oligonucleotide Rantes sense
cgctgtcatcctcattgcta

SEQ ID NO: 10 - DNA, 20 bp, artificial
  oligonucleotide Rantes antisense
gcacttgccactggtgtaga

(19) United States
(12) Patent Application Publication
(10) Pub. No.: US 2007/0149469 A1
(43) Pub. Date: Jun. 28, 2007

(54) Medical use of tbk-1 or of inhibitors thereof

(76) Inventor: Christian Korherr, Martinsried (DE)

Correspondence Address: CLARK & ELBING LLP, 101 FEDERAL STREET, BOSTON, MA, 02110, US

(21) Appl. No.: 10/574,306

(22) PCT Filed: Oct. 1, 2004
(86) PCT No.: PCT/EP04/10996

§ 371 Date: Aug. 7, 2006

Related U.S. Application Data
(60) Provisional application No. 60/508,100, filed on Oct. 2, 2003.

(51) Int. Cl. A61K 48/00

(52) U.S. Cl. 514/44

(57) ABSTRACT

The present invention relates to a novel angiogenic factor, TBK-1, we well as to inhibitors thereof and to their use in pharmaceutical or diagnostic compositions.

The present invention relates to the medical use of TBK-1 or of inhibitors thereof. Especially, the present invention relates to the use of these molecule in promoting or inhibiting angiogenesis.

Angiogenesis, the growth of new capillaries from pre-existing ones, is critical for normal physiological functions in adults [Carmeliet, P., Mechanisms of angiogenesis and arteriogenesis. Nat Med, 2000 6 (4) 389-95]. Abnormal angiogenesis can lead to impaired wound healing, poor tissue regeneration in ischemic conditions, cyclical growth of the female reproductive system, and tumor development [Carmeliet, P. and R. K. Jain, Angiogenesis in cancer and other diseases].

Promotion of angiogenesis is desirable in situations where vascularization is to be established or extended, for example after tissue or organ transplantation, or to stimulate establishment of collateral circulation in tissue infarction or arterial stenosis. The angiogenic process is highly complex and involves the maintenance of the endothelial cells in the cell cycle, degradation of the extracellular matrix, migration and invasion of the surrounding tissue and finally, tube formation. Because of the crucial role of angiogenesis in so many physiological processes, there is a need to identify and characterize factors which will promote angiogenesis.

The administration of growth factors such as VEGF-A and FGF-2 has been considered as a possible approach for the therapeutic treatment of ischemic disorders.

VEGF is an endothelial cell-specific mitogen and an angiogenesis inducer that is released by a variety of tumor cells and expressed in human tumor cells in situ.

However, both animal studies and early clinical trials with VEGF angiogenesis have encountered severe problems [Carmeliet, Nat Med, 2000 6 1102-3; Yancopoulos et al., However, both animal studies and early clinical trials with VEGF angiogenesis have encountered severe problems [Carmeliet, Nat Med, 2000 6 1102-3; Yancopoulos et al., Nature, 2000 407 242-8; Veikkola et al., Semin Cancer Biol 1999 9 211-20; Dvorak et al., Semin Perinatol 2000 24 75-8; Lee et al., Circulation, 2000 102 898-901]. VEGF-A stimulated microvessels are disorganized, sinusoidal and dilated, much like those found in tumors [Lee et al., Circulation 2000 102 898-901; and Springer et al., Mol. Cell. 1998 2 549-559]. Moreover, these vessels are usually leaky, poorly perfused, torturous and likely to rupture and regress. Thus, these vessels have limited ability to improve the ischemic conditions. In addition, the leakage of blood vessels induced by VEGF-A (also known as Vascular Permeability Factor) could cause cardiac oedema that leads to heart failure.

VEGF not only stimulates vascular endothelial cell proliferation, but also induces vascular permeability and angiogenesis. Angiogenesis, which involves the formation of new blood vessels from preexisting endothelium, is an important component of a variety of diseases and disorders including tumor growth and metastasis, rheumatoid arthritis, psoriasis, atherosclerosis, retinopathy, hemangiomas, immune rejection of transplanted tissues, and chronic inflammation.

In the case of tumor growth, angiogenesis appears to be crucial for the transition from hyperplasia to neoplasia, and for providing nourishment to the growing solid tumor. [Folkman, et al., Nature 339:58 (1989)]. Angiogenesis also allows tumors to be in contact with the vascular bed of the host, which may provide a route for metastasis of the tumor cells. Evidence for the role of angiogenesis in tumor metastasis is provided, for example, by studies showing a correlation between the number and density of microvessels in histologic sections of invasive human breast carcinoma and actual presence of distant metastases. [Weidner, et al., New Engl. J. Med. 324:1 (1991)].

In view of the importance of angiogenesis in various diseases, there is a continuous need for means interfering with angiogenesis. Therefore, the problem underlying the present invention resides in providing such means.

In the context of the present invention, it has been surprisingly found that TBK-1 expression in human and animal cells induces the production of a proangiogenic factor. Furthermore, it has been found that TBK-1 exhibits a proliferation inducing activity which is specific for endothelial cells. Finally, it has been surprisingly shown that TBK-1 inhibitors, especially siRNA, are able to inhibit VEGF expression (see Example 5).

TBK-1 (tank binding kinase 1) is a homologue of IKK-1 and IKK-2 (Kishore, N. et al, J. Biol. Chem. 277:13840, WO 00/73469, US 2003/0143540) and is known to be involved in inflammatory and immunologic processes. Furthermore, it is known that TBK-1 plays a role in NF-κB-activation, a transcription factor which is involved in many physiological processes like immunologic and inflammatory responses (Matsuda, A. et al., Oncogene 22:3307). It is activated e.g. by TNFalpha, IL1, LPS and various growth factors.

The protein sequence of human TBK-1 and the corresponding nucleic acid sequence are given in SEQ ID NO: 1 and 2, respectively. A role of TBK-1 in angiogenic processes has not been suggested in the art.

Consequently, according to one aspect of the invention, the problem is solved by the use of a nucleic acid encoding TBK-1 or a functional active derivative thereof for the preparation of a pharmaceutical composition for the treatment of diseases with disturbed angiogenesis, especially ischemic or dental diseases, smoker's leg and diabetic ulcers or for the stimulation of wound healing.

In the context of the present invention, the term “TBK-1” relates first to a protein with a sequence as shown in SEQ ID NO: 2. In a further aspect this term further relates to functional active derivatives of the protein as shown in SEQ ID NO: 2.

The term “functional active derivative” of a polypeptide within the meaning of the present invention refers to polypeptides which have a sequence homology, in particular a sequence identity, of about at least 25%, preferably about 40%, in particular about 60%, especially about 70%, even more preferred about 80%, in particular about 90% and most preferred of 98% with the polypeptide. Such derivatives are e.g. the polypeptide homologous to TBK-1, which originate from organisms other than the TBK-1 according to SEQ ID NO: 2. Other examples of derivatives are polypeptides which are encoded by different alleles of the gene, of different individuals, in different organs of an organism or in different developmental phases. Functional active derivatives preferably also include naturally occurring mutations, particularly mutations that quantitatively alter the activity of the peptides encoded by these sequences. Further, such variants may preferably arise from differential splicing of the encoding genes.

“Sequence identity” refers to the degree of identity (% identity) of two sequences, that in the case of polypeptides can be determined by means of for example BLASTP 2.2.5 and in the case of nucleic acids by means of for example BLASTN 2.2.6, wherein the low complexity filter is set on and BLOSUM is 62 (Altschul et al., 1997, Nucleic Acids Res., 25:3389-3402).

“Sequence homology” refers to the similarity (% positives) of two polypeptide sequences determined by means of for example BLASTP 2.0.1 wherein the Filter is set on and BLOSUM is 62 (Altschul et al., 1997, Nucleic Acids Res., 25:3389-3402).

Nucleic acids encoding functional active derivatives can be isolated by using human TBK-1 gene sequences in order to identify homologues with methods known to a person skilled in the art, e.g. through PCR amplification or hybridization under stringent conditions (e.g. 60° C. in 2.5×SSC buffer followed by several washing steps at room temperature concentration) with suitable probes derived from e.g. the human TBK-1 sequences according to standard laboratory methods (Current Protocols, John Wiley & Sons, Inc., New York (2003)).

“Functional active derivative” refers to a polypeptide that has essentially the biological function(s) as the corresponding protein. In the case of TBK-1, this may be the expression of a specific angiogenic activity as demonstrated in Example 1. Therefore, the term “functional active derivative” may also refer to a polypeptide which is responsible for the specific induction of endothelial cell proliferation. A test for the determination of the angiogenic activity induced by a putative TBK-1 derivative is also demonstrated in Example 1.

Furthermore, the term “Functional active derivative” may refer to the ability to induce the expression of VEGF as shown in Example 2.

A preferred embodiment for a nucleic acid encoding TBK-1 is given in SEQ ID NO: 1.

As demonstrated for the first time in the context of the present invention, TBK-1 is an important angiogenic factor. This enables the use of a nucleic acid encoding TBK-1 in therapy.

The administration of the nucleic acid encoding TBK-1 may be effected either as recombinant protein or by gene transfer either as naked DNA or in a vector [Komowski R, Fuchs S, Leon M B, Epstein S E, Delivery strategies to achieve therapeutic myocardial angiogenesis, Circulation, 2000 101 (4) 454-8; Simons M, Bonow R O, Chronos N A, Cohen D J, Giordano F J, Hammond H K, et al., Clinical trials in coronary angiogenesis: issues, problems, consensus: An expert panel summary, Circulation, 2000 102 (11) E73-86; and Isner J M, Asahara T, Angiogenesis and vasculogenesis as therapeutic strategies for postnatal neovascularization, J Clin Invest, 1999 103 (9) 1231-36]. If desired, regulatable vectors may be used as described in Ozawa et al, Annu Rev Pharmacol. & Toxicol, 2000 40 295-317.

Administration may be parenterally, intravenously, dermally, intradermally, intracutaneously, percutaneously, subcutaneously, topically or transdermally.

Alternatively, the nucleic acid can be administered by catheterbased myocardial gene transfer. In this technique, a steerable, deflectable 8F catheter incorporating a 27 gauge needle is advanced percutaneously to the left ventricular myocardium. A total dose of 200 ug/kg is administered as 6 injections into the ischemic myocardium (total, 6.0 mL). Injections are guided by NOGA left ventricular electromechanical mapping. See Vale, P. R., et al., Randomized, single-blind, placebo-controlled pilot study of catheter-based myocardial gene transfer for therapeutic angiogenesis using left ventricular electromechanical mapping in patients with chronic myocardial ischemia, Circulation, 2001 103 (17) 2138-43.

Another possibility is the injection of a TBK-1 plasmid in the muscles of an ischemic limb in accordance with procedures described in Simovic, D., et al., Improvement in chronic ischemic neuropathy after intramuscular phVEGF165 gene transfer in patients with critical limb ischemia, Arch Neurol, 2001 58 (5) 76168.

Still another technique for effective administration is by intra-arterial gene transfer of the gene using adenovirus and replication defective retroviruses as described for VEGF in Baumgartner I and Isner J M, Somatic gene therapy in the cardiovascular system, Annu. Rev Physiol, 2001 63 427-50. An additional possibility for administering the nucleic acid is by intracoronary and intravenous administration (see Post, M. J., et al., Therapeutic angiogenesis in cardiology using protein formulations, Cardiovasc Res, 2001 49 522-31).

A still further possibility is to use ex vivo expanded endothelial progenitor cells (EPCs) engineered to express TBK-1 for myocardial neovascularization as described in Kawamoto, A., et al., Therapeutic potential of ex vivo expanded endothelial progenitor cells for myocardial ischemia. Circulation, 2001 103 (5) 634-37.

Yet another technique which may be used to administer the nucleic acid is percutaneous adenovirus-mediated gene delivery to the arterial wall in injured atheromatous stented arteries. See, for example, Maillard, L., et al., Effect of percutaneous adenovirus-mediated Gax gene delivery to the arterial wall in double-injured atheromatous stented rabbit iliac arteries, Gene Ther, 2000 7 (16) 1353-61; and Laham R J, Simons M, and Sellke F, Gene transfer for angiogenesis in coronary artery disease, Annu Rev Med, 2001 52 485-502.

In one advantageous aspect of the invention, a therapeutically effective dose of the nucleic acid is administered by bolus injection of the active substance into ischemic tissue, e.g. heart or peripheral muscle tissue. The effective dose will vary depending on the weight and condition of the ischemic subject and the nature of the ischemic condition to be treated. It is considered to be within the skill of the art to determine the appropriate dosage for a given subject and condition. Furthermore, the pharmaceutical composition can be administered in further conventional manners, e.g. by means of the mucous membranes, for example the nose or the oral cavity, in the form of depositories implanted under the skin, by means of injections, infusions or gels which contain the medicaments according to the invention. It is further possible to administer the medicament topically and locally, if appropriate, in the form of liposome complexes. Furthermore, the treatment can be carried out by means of a transdermal therapeutic system (TTS), which makes possible a temporally controlled release of the medicaments. TTS are known for example, from EP 0 944 398 A1, EP 0 916 336 A1, EP 0 889 723 A1 or EP 0 852 493 A1.

In accordance with another aspect of the invention, the nucleic acid is administered by continuous delivery, e.g., using an osmotic minipump, until the patient is able to selfmaintain a functional vascular network.

In another advantageous aspect within the scope of the invention, the nucleic acid is effectively administered to an ischemic subject by contacting ischemic tissue with a viral vector, e.g. an adenovirus vector, containing a polynucleotide sequence encoding the protein operatively linked to a promoter sequence.

The nucleic acid may also be effectively administered by implantation of a micropellet impregnated with active substance in the direct vicinity of ischemic tissue.

For the production of the pharmaceutical compositions of the invention, the molecules of the present invention are usually formulated with suitable additives or auxiliary substances, such as physiological buffer solution, e.g. sodium chloride solution, demineralized water, stabilizers, such as protease or nuclease inhibitors, preferably aprotinin, ε-aminocaproic acid or pepstatin A or sequestering agents such as EDTA, gel formulations, such as white vaseline, low-viscosity paraffin and/or yellow wax, etc. depending on the kind of administration.

Suitable further additives are, for example, detergents, such as, for example, Triton X-100 or sodium deoxycholate, but also polyols, such as, for example, polyethylene glycol or glycerol, sugars, such as, for example, sucrose or glucose, zwitterionic compounds, such as, for example, amino acids such as glycine or in particular taurine or betaine and/or a protein, such as, for example, bovine or human serum albumin. Detergents, polyols and/or zwitterionic compounds are preferred.

The physiological buffer solution preferably has a pH of approx. 6.0-8.0, especially a pH of approx. 6.8-7.8, in particular a pH of approx. 7.4, and/or an osmolarity of approx. 200-400 milliosmol/liter, preferably of approx. 290-310 milliosmol/liter. The pH of the medicament is in general adjusted using a suitable organic or inorganic buffer, such as, for example, preferably using a phosphate buffer, tris buffer (tris(hydroxymethyl)aminomethane), HEPES buffer ([4-(2-hydroxyethyl)piperazino]ethanesulphonic acid) or MOPS buffer (3-morpholino-1-propanesulphonic acid). The choice of the respective buffer in general depends on the desired buffer molarity. Phosphate buffer is suitable, for example, for injection and infusion solutions.

Injection solutions are in general used if only relatively small amounts of a solution or suspension, for example about 1 to about 20 ml, are to be administered to the body. Infusion solutions are in general used if a larger amount of a solution or suspension, for example one or more litres, are to be administered. Since, in contrast to the infusion solution, only a few millilitres are administered in the case of injection solutions, small differences from the pH and from the osmotic pressure of the blood or the tissue fluid in the injection do not make themselves noticeable or only make themselves noticeable to an insignificant extent with respect to pain sensation. Dilution of the formulation according to the invention before use is therefore in general not necessary. In the case of the administration of relatively large amounts, however, the formulation according to the invention should be diluted briefly before administration to such an extent that an at least approximately isotonic solution is obtained. An example of an isotonic solution is a 0.9% strength sodium chloride solution. In the case of infusion, the dilution can be carried out, for example, using sterile water while the administration can be carried out, for example, via a so-called bypass.

Within the present invention, subjects which may be treated or diagnosed include animals, preferably mammals and humans, dead or alive. These patients suffer from the diseases as mentioned above.

The diseases mentioned above are all characterised by a disturbed angiogenesis and therefore a nucleic acid encoding TBK-1 leads to a significant improvement in these diseases.

With respect to the wound healing of fractures, the nucleic acid immobilised to a matrix can be administered directly into the site of fracture to promote the angiogenesis and wound healing. As matrices can be used ceramic matrices or bonemeal on which the protein is immobilised. Slow release formulations to have the factor locally enriched can be used as well.

In the context of the present invention, it could be shown that TBK-1 is a strong angiogenic factor. Therefore, in a preferred embodiment, the nucleic acid encoding TBK-1 induces the formation of vascular vessels.

As it can be taken from Example 2, a nucleic acid encoding TBK-1 is able to induce the production of VEGF. Therefore, in a preferred embodiment of the use of the present invention, the nucleic acid induces the production of VEGF.

The invention further includes a method for the treatment of a patient in need of such treatment, wherein an effective amount of a nucleic acid encoding TBK-1 is administered to the patient.

With respect to the preparation of this pharmaceutical composition, its administration and other embodiments the same apply as defined above.

As it is shown in Example 4 for colon cancer, the expression of TBK-1 is corrugated with VEGF. Consequently, TBK-1 or a nucleic acid encoding it can be used as diagnostic agents. Furthermore, TBK-1 detection is more specific than that of VEGF, since TBK-1 is located in the cell and therefore its expression can be exactly correlated with the production cell. In contrast, VEGF is a serum factor which means that it is more difficult to correlate its expression with its production cell.

The invention therefore relates to the use of
a) TBK-1,
b) a functional active derivative thereof,
c) a nucleic acid encoding TBK-1, and/or
d) means for the detection of the molecules of sections a), b), c) or d)
   for the preparation of a diagnostic agent for the diagnosis of ischemic or dental diseases, smoker's leg and diabetic ulcers, wound healing disorders, cancer, hyperplasia, tumor progression, rheumatoid arthritis, psoriasis, artherosclerosis, retinopathy, osteoarthritis, endometriosis and/or chronic inflammation.

This diagnostic agent may be appropriately combined with additional carriers or diluents or other additives which are suitable in this context With respect to these agents, the same apply as defined above for the pharmaceutical composition of the invention.

The proteins or nucleic acids may be prepared as defined above.

Within the meaning of the present invention, means of detecting TBK-1 or a function derivative thereof include antibodies which can e.g. applied in Western Blotting, Immunohistochemistry, ELISA or functional assays for the proteins (Current Protocols, John Wiley & Sons, Inc. (2003)).

Means for detecting the nucleic acids as defined above include other nucleic acids being capable of hybridizing with the nucleic acids e.g. in Southern Blots or Northern Blots as well as during In Situ Hybridization (Current Protocols, John Wiley & Sons, Inc. (2003)).

Angiogenesis is generally a phenomenon which occurs in later tumor stages. Since TBK-1 is an angiogenic factor, it represents therefore a marker for later tumor stages, i.e. for tumors which have already achieved a malignant state. Furthermore, since TBK-1 is an important angiogenic factor, its lack is indicative for the diseases disclosed above.

For example, TBK-1 or functional active derivatives thereof may be detected in the tumor tissue via immunohistochemistry. Nucleic acids encoding these molecules, e.g. mRNA, may be detected using quantitative PCR.

In several diseases as mentioned below, an aberrant angiogenesis contributes the clinical symptoms or is even the reason for these symptoms. The present invention relates to TBK-1, which is an important inducer of angiogenesis, e.g. in tumors. Therefore, the inhibition of TBK-1 results in inhibition of angiogenesis which will result in the treatment of these diseases.

In a further aspect, the present invention therefore relates to the use of a TBK-1 inhibitor for the preparation of a pharmaceutical composition for the treatment of a disease or diseases with increased angiogenesis, especially cancer, hyperplasia, rheumatoid arthritis, psoriasis, artherosclerosis, retinopathy, osteoarthritis, endometriosis and/or chronic inflammation.

According to the present invention the term “inhibitor” refers to a biochemical or chemical compound which preferably inhibits or reduces the angiogenic activity of TBK-1. This can e.g. occur via suppression of the expression of the corresponding gene. The expression of the gene can be measured by RT-PCR or Western blot analysis.

Examples of such TBK-1 inhibitors are binding proteins or binding peptides directed against TBK-1, in particular against the active site of TBK-1, and nucleic acids directed against the TBK-1 gene. Preferably, the inhibitor binds to the ATP-binding site of the kinase domain of TBK-1

In a preferred embodiment, the inhibitor of the invention is selected from the group consisting of antisense oligonucleotides, antisense RNA, siRNA, and low molecular weight molecules (LMWs).

LMWs are molecules which are not proteins, peptides antibodies or nucleic acids, and which exhibit a molecular weight of less than 5000 Da, preferably less than 2000 Da, more preferably less than 2000 Da, most preferably less than 500 Da. Such LMWs may be identified in High-Through-Put procedures starting from libraries. Such methods are known in the art. They preferably bind to the ATP-binding site of the kinase domain of TBK-1.

Nucleic acids which may inhibit TBK-1 activity may be double-stranded or single stranded DNA or RNA which, for example, inhibit the expression of the TBK-1 gene or the activity of TBK-1 and include, without limitation, antisense nucleic acids, aptamers, siRNAs (small interfering RNAs) and ribozymes.

The nucleic acids, e.g. the antisense nucleic acids or siRNAs, can be synthesized chemically, e.g. in accordance with the phosphotriester method (see, for example, Uhlmann, E. & Peyman, A. (1990) Chemical Reviews, 90, 543-584). Aptamers are nucleic acids which bind with high affinity to a polypeptide, here TBK-1 or derivatives thereof. Aptamers can be isolated by selection methods such as SELEX (see e.g. Jayasena (1999) Clin. Chem., 45, 1628-50; Klug and Famulok (1994) M. Mol. Biol. Rep., 20, 97-107; U.S. Pat. No. 5,582,981) from a large pool of different single-stranded RNA molecules. Aptamers can also be synthesized and selected in their mirror-image form, for example as the L-ribonucleotide (Nolte et al. (1996) Nat. Biotechnol., 14, 1116-9; Klussmann et al. (1996) Nat. Biotechnol., 14, 1112-5). Forms which have been isolated in this way enjoy the advantage that they are not degraded by naturally occurring ribonucleases and, therefore, possess greater stability.

Nucleic acids may be degraded by endonucleases or exonucleases, in particular by DNases and RNases which can be found in the cell. It is, therefore, advantageous to modify the nucleic acids in order to stabilize them against degradation, thereby ensuring that, a high concentration of the nucleic acid is maintained in the cell over a long period of time (Beigelman et al. (1995) Nucleic Acids Res. 23:3989-94; WO 95/11910; WO 98/37240; WO 97/29116). Typically, such a stabilization can be obtained by introducing one or more internucleotide phosphorus groups or by introducing one or more non-phosphorus internucleotides.

Suitable modified internucleotides are compiled in Uhlmann and Peyman (1990), supra (see also Beigelman et al. (1995) Nucleic Acids Res. 23:3989-94; WO 95/11910; WO 98/37240; WO 97/29116). Modified internucleotide phosphate radicals and/or non-phosphorus bridges in a nucleic acid which can be employed in one of the uses according to the invention contain, for example, methyl phosphonate, phosphorothioate, phosphoramidate, phosphorodithioate and/or phosphate esters, whereas non-phosphorus internucleotide analogues contain, for example, siloxane bridges, carbonate bridges, carboxymethyl esters, acetamidate bridges and/or thioether bridges. It is also the intention that this modification should improve the durability of a pharmaceutical composition which can be employed in one of the uses according to the invention.

The use of suitable antisense nucleic acids is further described e.g. in Zheng and Kemeny (1995) Clin. Exp. Immunol., 100, 380-2; Nellen and Lichtenstein (1993) Trends Biochem. Sci., 18, 419-23, Stein (1992) Leukemia, 6, 697-74 or Yacyshyn, B. R. et al. (1998) Gastroenterology, 114, 1142).

The production and use of siRNAs as tools for RNA interference in the process to down regulate or to switch off gene expression, here TBK-1 gene expression, is e.g. described in Elbashir, S. M. et al. (2001) Genes Dev., 15, 188 or Elbashir, S. M. et al. (2001) Nature, 411, 494. Preferably, siRNAs exhibit a length of less than 30 nucleotides, wherein the identity stretch of the sense strang of the siRNA is preferably at least 19 nucleotides.

Ribozymes are also suitable tools to inhibit the translation of nucleic acids, here the TBK-1 gene, because they are able to specifically bind and cut the mRNAs. They are e.g. described in Amarzguioui et al. (1998) Cell. Mol. Life Sci., 54, 1175-202; Vaish et al. (1998) Nucleic Acids Res., 26, 5237-42; Persidis (1997) Nat. Biotechnol., 15, 921-2 or Couture and Stinchcomb (1996) Trends Genet., 12, 510-5.

Thus, the nucleic acids described can be used to inhibit or reduce the expression of the TBK-1 genes in the cells both in vivo and in vitro and consequently act as a TBK-1 inhibitor in the sense of the present invention. A single-stranded DNA or RNA is preferred for the use as an antisense oligonucleotide or ribozyme, respectively.

For the context of these diseases, TBK-1 inhibition aims at preventing the formation of vascular vessels which support the diseased tissue. This, in turn, will reduce the amount of diseased or malignant cells (e.g. cancer cells).

The pharmaceutical composition may be prepared and administered as discussed above.

The analysis in colon of TBK-1 expression and VEGF expression, as shown in FIG. 6, shows that TBK-1 is regulated in a similar manner as VEGF. A correlation between TBK-1 and VEGF expression is found both in normal colon tissue and colon cancer. This is a further hint that TBK-1 regulates the expression of VEGF. Consequently, therapeutic means that are capable of reducing the expression and/or activity of TBK-1 can in turn cause reduced expression and hence reduced activity of VEGF.

Therefore, the inhibitor of the invention may act through the inhibition of the production of VEGF. Therefore, in a preferred embodiment of this use of the present invention, the inhibitor inhibits the production of VEGF.

As it is shown in Example 3, TBK-1 expression is upregulated under hypoxic conditions. It is known in the art that during the growth of solid tumors, often hypoxic conditions are found, which in turn result in the induction of new vascular vessels. TBK-1 may be an important factor in this physiological process. In turn, inhibition of TBK-1 function may result in maintaining the hypoxic conditions in the tumor, resulting in a suppression of tumor growth or even in a regression of tumor size.

Therefore, in a preferred embodiment of the use of the invention, the inhibitor prevents the formation of vascular vessels in the tumor tissue.

According to a preferred embodiment, the disease is cancer, preferably selected from the group consisting of brain cancer, pancreas carcinoma, stomach cancer, colon carcinoma, skin cancer, especially melanoma, bone cancer, kidney carcinoma, liver cancer, lung carcinoma, ovary cancer, mamma carcinoma, uterus carcinoma, prostate cancer and testis carcinoma.

The invention further includes a method for the treatment of a patient in need of such treatment, wherein an effective amount of an inhibitor of TBK-1 or of a functional active derivative thereof is administered to the patient.

With respect to the preparation of this pharmaceutical composition, its administration and other embodiments the same apply as defined above.

The invention further relates to a method for the identification of an anti-cancer drug, wherein
a) a potential TBK-1 interactor is brought into contact with TBK-1 or a functional derivative thereof, and
b) binding of the potential interactor to TBK-1 or the functional derivative thereof is determined, and
c) the anti-angiogenic capacity of the potential interactor is determined.

In this method of the invention, in general, TBK-1 or the corresponding gene are provided e.g. in an assay system and brought directly or indirectly into contact with a test compound, in particular a biochemical or chemical test compound. Then, the influence of the test compound on TBK-1 or the corresponding gene is measured or detected by measuring whether the TBK-1 phenotype is reversed by addition of the potential inhibitor. Thereafter, suitable inhibitors can be analyzed and/or isolated. For the screening of compound libraries, the use of high-throughput assays are preferred which are known to the skilled person or which are commercially available.

Suitable assays may be based on the gene expression of TBK-1 or on the physiological activity of TBK-1, i.e. the angiogenic properties.

For example, the following assay may be used for the identification of an inhibitor of the invention:
transfection of TBK-1 into HEK293 cells (see also sequence listing)
transfer of supernatants of HEK 293 cells onto HUVEC cells (as described for the screen in example 1)
addition/incubation of HUVEC cells with LMW (low molecular weight) compound library or other potential inhibitors
screening for inhibition of proliferating activity (reversion of phenotype)
definition of lead structures
analysis of specificity: inhibition of TBK-1

The experimental steps transfection of 293 cells, transfer of supernatant onto HUVEC cells and screening for proliferation or inhibition of proliferation, respectively, can be carried out according to Example 1.

In a preferred embodiment, in the method of the invention the anti-angiogenic capacity is measured by measuring the inhibition of VEGF production.

Preferably, in the method of the invention the potential interactor is provided in the form of a chemical compound library.

According to the present invention the term “chemical compound library” refers to a plurality of chemical compounds that have been assembled from any of multiple sources, including chemically synthesized molecules and natural products, or that have been generated by combinatorial chemistry techniques.

In a further preferred embodiment, in the method of the invention the chemical compound library consists of a group of molecules or substances that bind to the ATP binding site of the kinase domain of TBK-1.

More preferred, the method of the invention is carried out on an array. Methods for preparing such arrays using solid phase chemistry and photolabile protecting groups are disclosed, for example, in U.S. Pat. No. 5,744,305. These arrays can also be brought into contact with test compound or compound libraries and tested for interaction, for example binding or changing conformation.

In another embodiment of the present invention, the method is carried out in form of a high-through put screening system. In such a system advantageously the screening method is automated and miniaturized, in particular it uses miniaturized wells and microfluidics controlled by a robot.

The following Figures and Examples are intend to illustrate further the invention without limiting it.

### SHORT DESCRIPTION OF THE FIGURES

FIG. 1 indicates proliferation of HUVEC following transfer of supernatants from transfected HEK 293 cells. The relative fluorescence units (RFU) are given as mean value from six independent experiments.

Vector represents the negative control resulting from transfection of the cloning vector pCMV6-XL into HEK 293 cells and measurement of Alamar Blue to determine background proliferative effect of the supernatant derived from HEK 293 cells. VEGF was derived from the same clone collection to ensure compatibility and compatibility of expression systems.

FIG. 2 indicates proliferation of normal human dermal fibroblasts (NHDF) following transfer of supernatants from transfected HEK 293 cells. The relative fluorescence units (RFU) are given as mean value from three independent experiments.

Vector represents the negative control resulting from transfection of the cloning vector pCMV6-XL into HEK 293 cells and measurement of Alamar Blue to determine background proliferative effect of the supernatant derived from HEK 293 cells. FGF-2 was derived from the same clone collection to ensure compatibility of expression systems. The figure demonstrates that TBK-1 was unable to stimulate NHDF proliferation to levels above empty vector controls.

FIG. 3 indicates proliferation of human microvascular endothelial cells (HMVEC) following transfer of supernatants from transfected HEK 293 cells. The relative fluorescence units (RFU) are given as mean value from three independent experiments.

Vector represents the negative control resulting from transfection of the cloning vector pCMV6-XL into HEK 293 cells and measurement of Alamar Blue to determine background proliferative effect of the supernatant derived from HEK 293 cells. VEGF was derived from the same clone collection to ensure compatibility of expression systems. The figure demonstrates that TBK-1 was able to stimulate HMVEC proliferation to levels above empty vector controls

FIG. 4 describes that HEK 293 cells transfected with TBK-1 produce VEGF. The OD values at 492 nm are given as mean value from three independent VEGF-ELISA experiments.

FIG. 5 indicates induced expression of TBK-1 in HEK 293 cells under hypoxic conditions simulated by incubation with CoCl₂. TBK-1 levels are presented relative to expression levels of G6PDH.

FIG. 6 shows the correlation between TBK-1 and VEGF expression in normal colon tissue compared to colon cancer tissue.

Total RNA from colon tissue (cancer and normal) was transcribed into cDNA and relative expression of TBK-1 and VEGF versus G6PDH was calculated after quantitative real-time PCR. A correlation between TBK-1 and VEGF expression in normal colon tissue can be shown. Correlation is also seen in colon cancer tissue, however less pronounced.

FIG. 7a shows the effect of TBK-1 RNAi on the expression of TBK-1 mRNA. The RNAi molecule reduces the expression of TBK-1 by more that 85%. TBK-1 knock down was verified by analysing relative expression levels of TBK-1 by quantitative RT-PCR compared to expression levels of G6PDH.

FIG. 7b shows the effect of RNAi-mediated TBK-1 inhibition on hypoxia-induced expression of VEGF in HEK293 cells. VEGF-expression levels were determined by analysing relative expression by quantitative RT-PCR compared to expression levels of G6PDH.

FIG. 8: Expression of TBK-1 in breast cancer vs. normal breast tissue

Staining for TBK-1 protein was strongly positive in the breast cancer tissue sample compared to normal tissue were staining was basically negative. Staining was performed as described above. The expression in tumor tissue was observed predominantly in the malignant cells.

In the normal tissue, glandular cells just display the (blue) conterstain, while the cancerous cells are detected by the specific antibody stain (left).

FIG. 9: Expression of TBK-1 in tumor vs normal tissue by quantitative RT-PCR Total RNA from colon, lung, prostate and breast tissue was transcribed into cDNA and relative expression of TBK-1 versus 18SrRNA was calculated after quantitative real-time PCR. Absolute expression levels have been analysed by quantitative real-time PCR for a panel of cDNAs from mammary gland and ovary tissue.

Overexpression of TBK-1 was observed in most colon, lung, prostate and breast cancer compared to normal tissue.

FIG. 10: Induction of Rantes by TBK-1

Total RNA from HEK293 cells transfected with TBK-1 or vector control was transcribed into cDNA and relative expression of Rantes versus. G6PDH was calculated after quantitative real-time PCR. Indicated is the relative induction of Rantes by TBK-1 compared to empty vector.

Induction of Rantes was observed by overexpression of TBK-1 in HEK293 cells.

FIG. 11: Specific inhibition of TBK-1 induced activity by low molecular compounds FIG. 11 shows inhibition of TBK-1 (assay 1) and TICAM (assay 2) induced proliferative activity of supernatants on HUVEC cells by indicated compounds. VEGF served as non-target control.

FIG. 12: Dose dependent inhibition of TBK-1 induced activity by a low molecular compound

FIG. 12 shows the dose dependent inhibition of TBK-1 with the compound PLX002-A10 PLX002-A10 showed a dose dependent inhibition of TBK-1 and TICAM induced activity.

### EXAMPLES

### Example 1

### Proliferation Inducing-Activity

An expression screen was conducted in order to isolate novel cDNAs that encode secreted proteins which stimulate endothelial cell proliferation. Plasmid DNAs were prepared on Xantos' proprietary high-throughput robot assembly according to standard Xantos protocols (see WO 03/014346).

To facilitate the production of the proteins encoded by individual cDNA clones, 2.2×10⁴ 293 HEK cells were seeded in 96-well tissue culture plates (Costar) in 100 μl DMEM medium containing 5% FCS (Invitrogen). Transfection of ca. 10000 cDNAs from a clone collection (Human Full-Length Clone Collection”, OriGene Technologies Inc., Rockville, Md., U.S.A.) on 293 cells was performed 24 hrs post seeding using calcium phosphate co-precipitation. Precipitates were removed after 4 hours and cells were switched to nutrient deficient DMEM (DMEM, 1.5% FCS, 1% Na-pyruvate, 1% Glutamine, 100 μg/ml gentamycin, 0.5 μg/ml amphotericin B). Human umbilical cord vein endothelial cells (HUVEC) were cultured in ECGM with supplements Promocell Heidelberg, single quotes) containing 1% serum, 50 μg/ml gentamycin, 0.4 μg/ml amphotericin B and 50 U/ml nystatin. HUVECS were plated at 2.5×10³ cells/well on day 3. Before transfer of supernatants on day 4, 90 μl of medium was removed, HUVECS were washed once with 200 μl of PBS, then 75 μl of nutrient deficient medium (ECBM, with supplements, Promocell, Heidelberg) containing 1 μg/ml hydrocortisol, 50 μg/ml gentamycin, 0.4 μg/ml amphotericin B and 50 U/ml nystatin was added following 25 μl of supernatants from the transfected 293 cells. Supernatants were incubated for 4 days on HUVEC cells. Read-out was performed using Alamar Blue (Biosource, Calif. USA). For each well of a 96 well plate, 11 μl of Alamar Blue reagent were mixed with 9 μl of ECBM and the resulting 20 μl were added directly to the HUVEC cells without removal of medium. Incubation was performed at 37° C. for 4 hours. Alamar Blue fluorescence was measured at 530 nm excitation and 590 nm emission.

As a positive control for proliferation of HUVECs, a supernatant from cells transfected with VEGF cDNA derived from the same clone collection was used.

Negative controls were supernatants from vector-transfected HEK 293 cells.

This screen led to the isolation of a cDNA which will be referred to as TANK-binding kinase 1 (TBK-1).

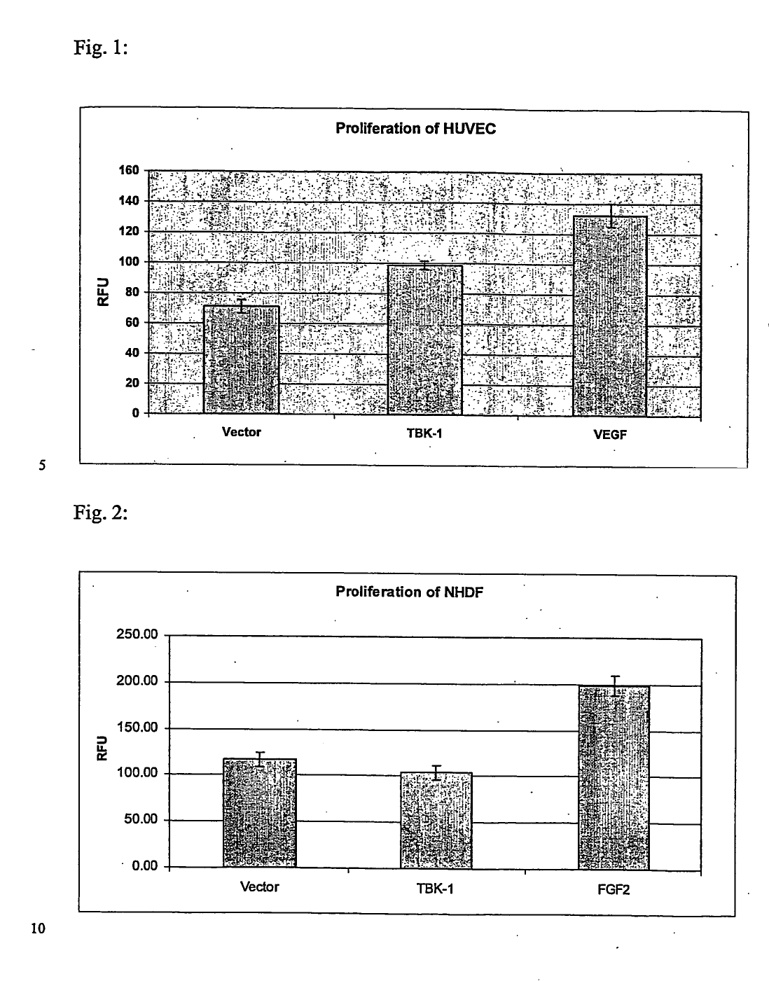

For the verification or specification of the proliferation-inducing activity of TBK-1, TBK-1 and controls were transfected into HEK293 cells and supernatants were transferred onto HUVEC as described for the screen above except that all manipulations were carried out manually. FIG. 1 shows the proliferation-inducing activity of TBK-1 in comparison to VEGF.

In order to investigate the cell type specificity of TBK-1, supernatants from transfected HEK 293 cells were also added to normal human dermal fibroblasts (NHDF). NHDF were seeded at 1,000 cells per well on 96-well tissue culture plates two days prior to the transfer in 100 μl complete Fibroblast Growth Medium (Promocell, Heidelberg). 24 h prior to the transfer the medium was changed to 100 μl Fibroblast Basal Medium (Promocell, Heidelberg) containing 75 μg/ml gentamycin, 50 ng/ml amphotericin B. After 25 μl of HEK 293 supernatant had been transferred, cells were incubated for 4 days and viable cell number was assessed by Alamar Blue reduction as above. FIG. 2 demonstrates that TBK-1 was unable to stimulate NHDF proliferation to levels above empty vector controls. However, the cells were clearly responsive to supernatants containing FGF-2. These results demonstrate that TBK-1 acts specifically on endothelial, but not fibroblast cells.

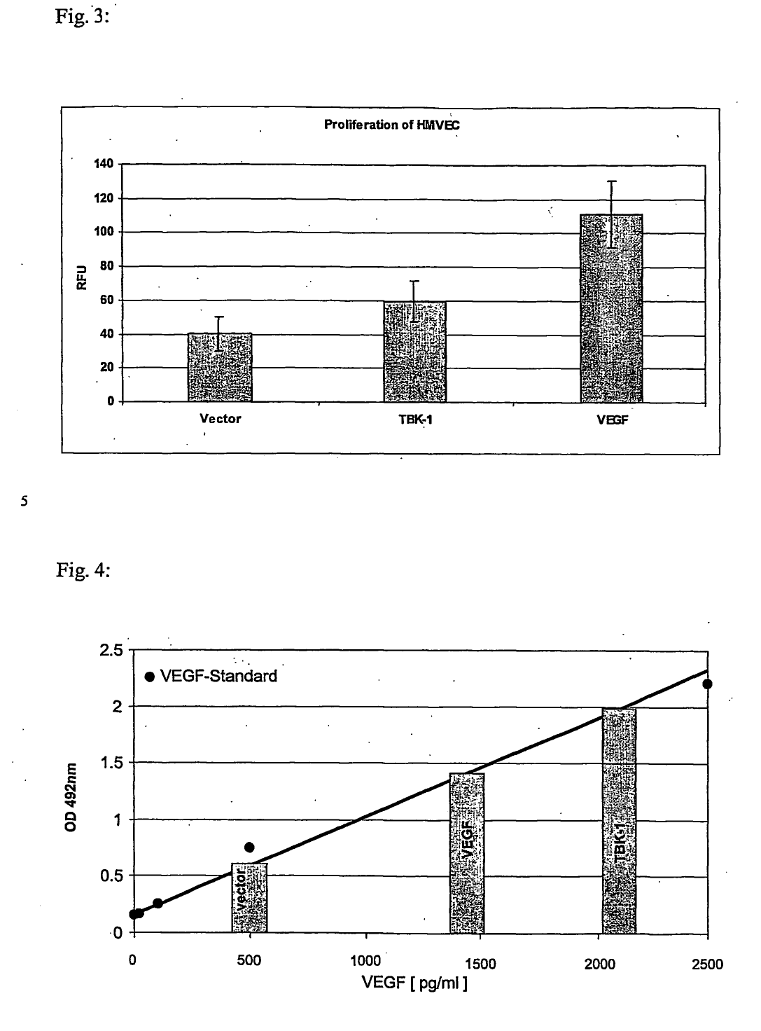

In order to investigate the cell type specificity of TBK-1, supernatants from transfected HEK 293 cells were also added to human microvascular endothelial cells (HMVEC). HMVECS were cultured in EBM with supplements (Cell Systems, St. Katharinen, single quotes). HMVECS were plated at 3.2×10³ cells/well on day 3. Before transfer of supernatants on day 4, 90 μl of medium was removed, HMVECS were washed once with 200 μl of PBS, then 75 μl of nutrient deficient medium (EBM, Cell Systems, St. Katharinen) containing, 2% FBS, 1 μg/ml hydrocortisol, 50 μg/ml gentamycin and 0.4 μg/ml amphotericin B was added following 25 μl of supernatants from the transfected 293 cells. Supernatants were incubated for 5 days on HMVEC cells. Read-out was performed using Alamar Blue (Biosource, California USA). For each well of a 96 well plate, 11 μl of Alamar Blue reagent were mixed with 9 μl of EBM and the resulting 20 μl were added directly to the HMVEC cells without removal of medium. Incubation was performed at 37° C. for 4 hours. Alamar Blue fluorescence was measured at 530 nm excitation and 590 nm emission. FIG. 3 demonstrates that TBK-1 was able to stimulate HMVEC proliferation to levels above empty vector controls.

The results of these analysis showed that proliferation-inducing activity of TBK-1 is specific on endothelial cells (EC and HMVEC) and not on fibroblast cells (NHDF).

### Example 2

### Induction of VEGF

To determine the mechanism by which TBK-1 leads to the proliferation of endothelial cells we tested the induction of VEGF by TBK-1. VEGF was measured in an ELISA specific for detection of hVEGF. 2×10⁴ HEK 293 cells were transfected in parallel with 0.28 μg of the indicated cDNAs (see FIG. 1) and grown in serum reduced culture medium (1.5% FCS). Concentration of hVEGF in the supernatant was determined 48 h after transfection according to the manufacturers protocol (PromoKine—Human VEGF ELISA Kit, PromoCell GmbH, Heidelberg, Germany). The empty vector pCMVSport6 was used as negative control. As positive control cells were transfected with an expression plasmid for hVEGF. Shown in FIG. 4 are means of 3 independent experiments which revealed that TBK-1 expression leads to the expression of VEGF.

The induction of hVEGF by TBK-1 is significantly higher compared to the vector control (˜3 fold). The concentration of hVEGF in supernatants of TBK-1 transfected cells is similar to cells transfected with the expression plasmid for hVEGF.

### Example 3

### Increased Expression of TBK-1 in HEK 293 Cells Under Hypoxic Conditions

VEGF expression takes place under hypoxic conditions. To analyse the effects of hypoxia on expression of TBK-1, we measured the expression levels of TBK-1 in RNAs and cDNAs from HEK 293 cells either untreated or incubated with medium containing 50 mM CoCl₂ for 24 hours. Expression levels were analysed by quantitative real-time PCR. Incubation with CoCl₂ is an accepted model for chemical induction of hypoxic conditions in cells.

cDNA was synthesized from 1 μg of total RNA in a volume of 20 μl using random hexamers as primer and AMV ReverseTranscriptase (Roche Diagnostics).

Real-time PCR was carried out using a LightCycler (Roche Diagnostics). Reactions were set up in microcapillary tubes using the following final concentrations: 0.5 μM each of TBK-1 sense (TTG AAG AGG AGA CAA CAA CAA GA) and TBK-1 antisense (CAT TCC ACC CAC CAC ATC T) primers, 3 mM MgCl₂, 1×SYBR Greenmaster mix and 2 μl of cDNA.

Cycling conditions for TBK-1 were as follows: denaturation (95° C. for 10 min), amplification and quantitation (95° C. for 10 s, 58° C. for 10 s and 72° C. for 13 s, with a single fluorescence measurement at the end of the 72° C. for 13 s segment) repeated 45 times. A melting curve program (55-95° C. with a heating rate of 0.1° C./s and continuous fluorescence measurement) and a cooling step to 40° C. followed.

For relative quantification the procedure was repeated for G6PDH mRNA as reference gene. Data were analyzed using LightCycler analysis software.

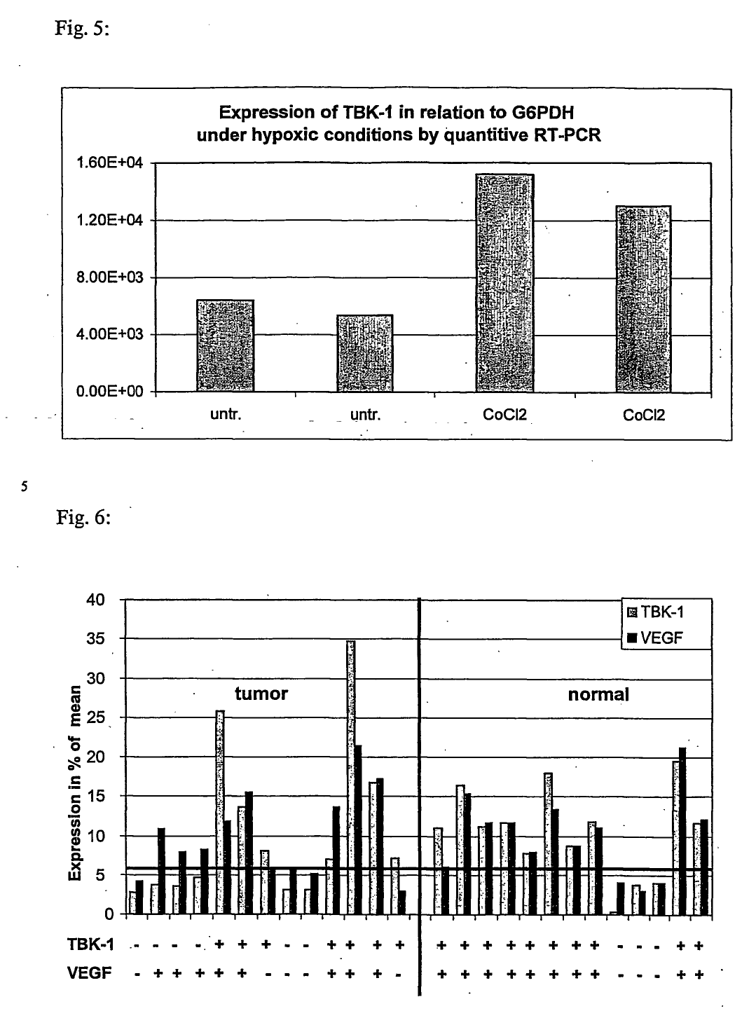

The results of our expression analysis revealed that the expression level of TBK-1 under hypoxic conditions is significantly higher compared to untreated cells. Thus the expression of TBK-1 is induced by hypoxic conditions in relation to G6PDH. (FIG. 5).

### Example 4

### Expression of TBK-1 in Colon Cancer Versus Normal Tissues Compared to VEGF

To analyse whether TBK-1 is regulated in a similar manner as VEGF in normal and tumour tissue, the expression levels of TBK-1 and VEGF in RNAs and cDNAs from human colon (normal and cancer) were analysed by quantitative real-time PCR.

cDNA was synthesized from 1 μg of total RNA in a volume of 20 μl using oligo (dT)₁₂ as primer and AMV ReverseTranscriptase (Roche Diagnostics).

Real-time PCR was carried out using a LightCycler (Roche Diagnostics). Reactions were set up in microcapillary tubes using the following final concentrations: 0.5 μM each of VEGF sense (CTT GCC TTG CTG CTC TAC CT) and VEGF antisense (GAT TCT GCC CTC CTC CTT CT) primers, 3 mM MgCl₂, 1×SYBR Greenmaster mix and 2 μl of cDNA. For TBK-1 0.5 μM each of TBK-1 sense (TTG AAG AGG AGA CAA CAA CAA GA) and TBK-1 antisense (CAT TCC ACC CAC CAC ATC T) primers were used.

Cycling conditions for VEGF were as follows: denaturation (95° C. for 10 min), amplification and quantitation (95° C. for 10 s, 58° C. for 10 s and 72° C. for 13 s, with a single fluorescence measurement at the end of the 72° C. for 13 s segment) repeated 45 times. A melting curve program (55-95° C. with a heating rate of 0.1° C./s and continuous fluorescence measurement) and a cooling step to 40° C. followed. Cycling conditions for TBK-1 were identical.

For relative quantification the procedure was repeated for G6PDH mRNA as reference gene. Data were analyzed using LightCycler analysis software.

The results of these analysis showed that the relative expression levels of TBK-1 are comparable to relative expression levels of VEGF (FIG. 6). A correlation between TBK-1 and VEGF expression in normal colon tissue is also seen in colon cancer however the correlation is less pronounced.

### Example 5

### Inhibition of VEGF Expression Under Hypoxic Conditions by RNAi Against TBK-1

If TBK-1 was to be causative for hypoxia induced expression of VEGF, or significantly involved in expression of VEGF, it should be possible to inhibit or reduce the hypoxia induced expression of VEGF by inhibiting the activity of TBK-1. To analyze this effect, D we inhibited the expression of TBK-1 utilizing TBK-1-specific RNAi molecules. The use of RNAi to inhibit gene expression and function is a principle that is well known to experts in the field (Elbashir, S. M. et al. (2001) Genes Dev., 15, 188 or Elbashir, S. M. et al. (2001) Nature, 411, 494).

HEK 293 cells were transfected with double stranded RNAi oligos specific for TBK-1 using siPORT Amine according to the manufacture's protocol (Ambion Europe LTD, Cambridgeshire, United Kingdom). Reactions were set up using the following final concentration: 100 nM each of pre-annealed siTBK-1 sense (GGA GAC AAC AAC AAG ACA Utt) (Seq ID No 3) and siTBK-1 antisense (AUG UCU UGU UGU UGU CUC Ctc) (Seq ID No 4) primers. 24 hours after transfection cells were incubated with culture medium containing 50 mM CoCl₂ as described in example 3. Under these conditions, increased expression of VEGF is observed and can be detected via VEGF ELISA (as shown in example 3).

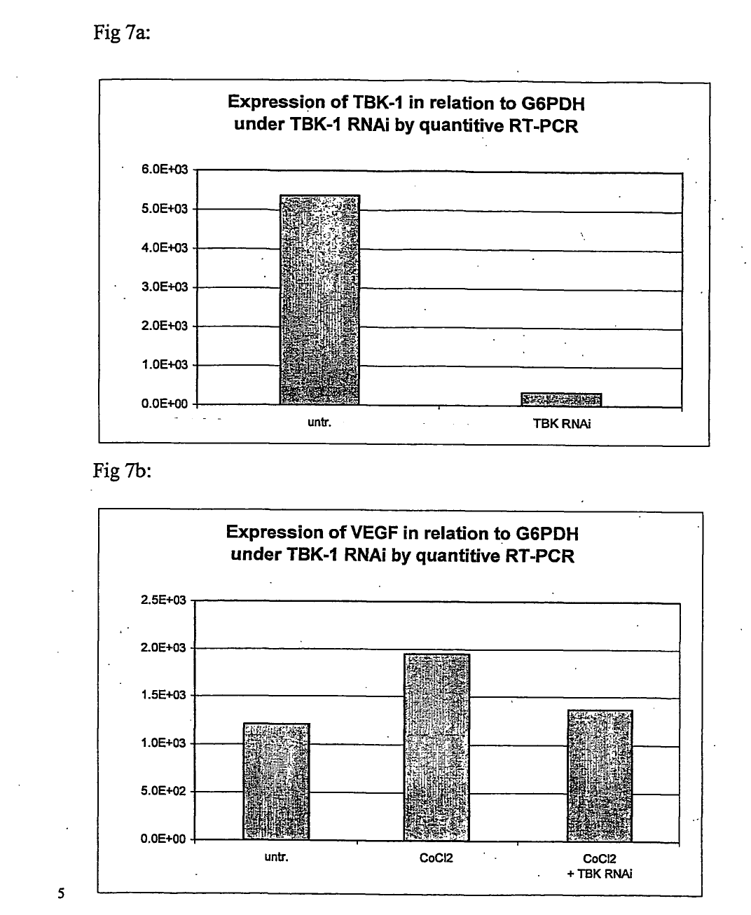

To determine the functionality of the RNAi molecule regarding inhibition of TBK-1 expression, relative expression levels of TBK-1 mRNAs were analysed by quantitative real-time PCR. Conditions for QPCR were identical to those described in example 3. Expression levels for VEGF mRNA were analyzed as described in example 4 with the exception that cDNA was synthesized using random hexamers as primers. The result of this experiment, shown in FIG. 7a, shows that the TBK-1 specific RNAi molecule inhibits the expression of TBK-1 by more that 85%.

The effect of application of this RNAi molecule was then analyzed in HEK 293 cells which were set under CoCl₂ induced hypoxia, as described in example 3. Without TBK-1 RNAi inhibition these cells express significantly increased amounts of VEGF mRNA under CoCl₂ exposure. Application of TBK-1 RNAi under identical conditions significantly reduces the induction of VEGF mRNA under hypoxia. These data are presented in FIG. 7b. Thus, inhibition of TBK-1 can be utilized to reduce the expression levels of VEGF and to counteract hypoxia induced expression of VEGF.

### Example 6

### Increased Expression of TBK-1 in Tumor Tissue

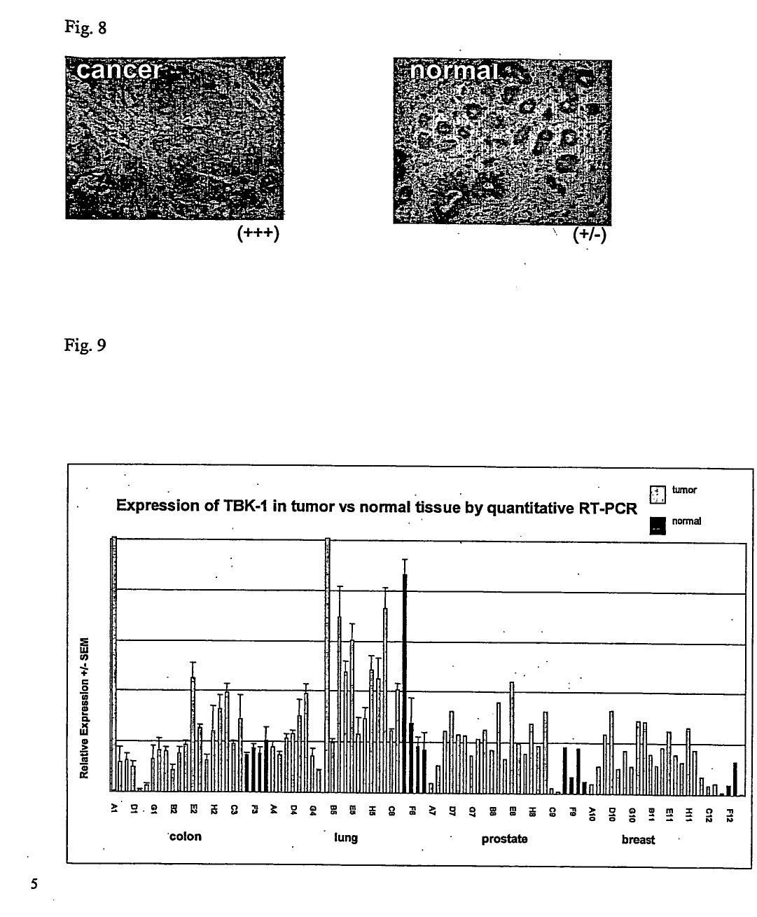

For the analysis of TBK-1 expression in tumor tissue, tissue samples of patients (normal and tumor tissue) were stained for TBK-1 protein using immunohistochemistry (IHC) and mRNA levels were measured using quantitative real-time PCR (QPCR) as described in example 3. Table 1 shows expression of TBK-1 in different solid tumors and corresponding normal tissues. FIG. 8 shows increased expression of TBK-1 in breast tumor compared to adjent normal tissue analysed by IHC as an example. FIG. 9 shows increased expression of TBK-1 in tumor tissue compared to normal tissue analysed by QPCR.

TABLE 1
Expression of TBK-1 in different normal and tumor tissue

| | Normal | | | |
|---|---|---|---|---|
| Tissue | (IHC) | Normal (QPCR) | Cancer (ICH) | Cancer (QPCR) |
| Breast | +/− | − | ++ | + |
| | | (mammary gland) | | |
| Colon | − | +/− | ++/+++ | + |
| Lung | − | + | + | ++ |
| Prostate | + | − | n.d. | + |

n.d.: not determined

Indicated tissue samples were either stained for TBK-1 protein by immunohistochemistry using anti-TBK-1 antibody (Calbiochem) or were analysed for TBK-1 RNA expression by QPCR. QPCR was performed as described in example 3. Immunostaining (Applied Phenomics, Estonia) was performed on whole body tissue arrays (core diameter 0.6 and 1.5 mm, paraformaldehyde fixed and paraffin-embedded material). Manual immunostaining using DAKO secondary reagents (DAKO Duet HRP kit) was performed using standard citrate/microwave pre-treatment. Unspecific binding of secondary reagents was prevented by biotin blocking. The results were be evaluated by experts in immunohistochemistry and a pathologist.

Normal tissue tested by IHC showed some positively for adrenal gland, pancreas, testis, thyroid, bone marrow, spleen, tonsils, salivary gland, liver, stomach, small intestine, kidney, oviduct, prostate, skin and lymph node and negative for brain, peripheral nerve, lung, myocard, aorta, vena cava, esophagus, colon, bladder, uterus, cervix, skeletal muscle and adipose tissue.

FIG. 9 shows the results of TBK-1 expression level in normal tissue and cancer samples via QPCR. For that expression levels of TBK-1 in RNAs and cDNAs from human colon (normal and cancer), lung (normal and cancer), prostate (normal and cancer) and breast (normal and cancer) were analysed by quantitative real-time PCR.

cDNA was synthesized from 1 μg of total RNA in a volume of 20 μl using random hexamers as primer and AMV ReverseTranscriptase (Roche Diagnostics). Real-time PCR was carried out using a LightCycler (Roche Diagnostics) as described in example 3. For relative quantification the procedure was repeated for 18S rRNA as reference gene. Data were analyzed using LightCycler analysis software.

The results indicates higher expression of human TBK-1 in most cancer versus normal tissues.

### Example 7

### TBK-1 Expression Induces Pro-Angiogenic Factors

Expression analysis of HEK 293 cells transfected with TBK-1 in comparison to cells transfected with vector as control were performed to analyse whether additional pro-angiogenic factors are induced by TBK-1. Therefore total RNA from transfected cells was analysed using Affymetrix Chip analysis. Besides several small inducible cytokines the known proliferative protein Rantes was induced by TBK-1. To verify this observation expression of Rantes was analysed in HEK293 cells transfected with TBK-1 using quantitative real-time PCR.

For these experiments cDNA was synthesized from 1 μg of total RNA in a volume of 20 μl using random hexamers as primer and AMV ReverseTranscriptase (Roche Diagnostics).

Real-time PCR was carried out using a LightCycler (Roche Diagnostics). For analysis of Rantes reactions were set up in microcapillary tubes using the following final concentrations: 1 μM each of Rantes sense (CGC TGT CAT CCT CAT TGC TA) and Rantes antisense (GCA CTT GCC ACT GGT GTA GA) primers, 2.5 μM MgCl₂, 1×SYBR Greenmaster mix and 0.2 μl of cDNA. Cycling conditions were as follows: denaturation (95° C. for 10 min), amplification and quantitation (95° C. for 10 s, 55° C. for 10 s and 72° C. for 13 s, with a single fluorescence measurement at the end of the 72° C. for 13 s segment) repeated 45 times. A melting curve program (55-95° C. with a heating rate of 0.1° C./s and continuous fluorescence measurement) and a cooling step to 40° C. followed.

For relative quantification the procedure was repeated for G6PDH RNA as reference gene. Data were analyzed using LightCycler analysis software.

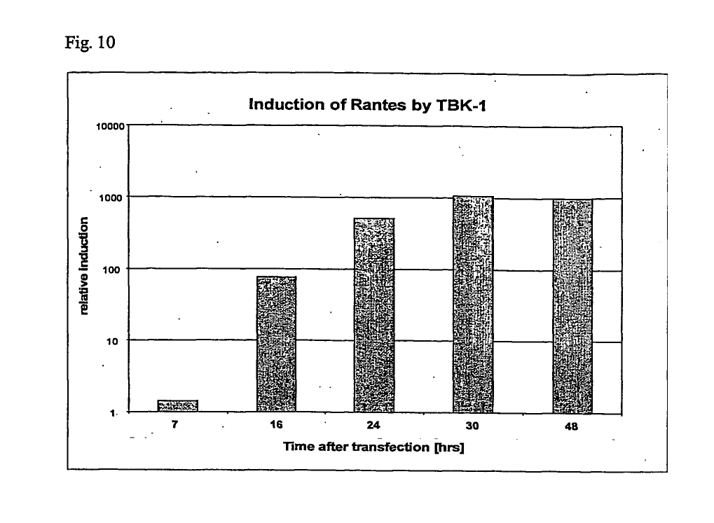

The results of these analyses are shown in FIG. 10 which indicates increased expression of Rantes in HEK293 cells transfected with TBK-1 compared to vector control.

### Example 8

### Inhibition of TBK-1 Activity by Chemical Compounds

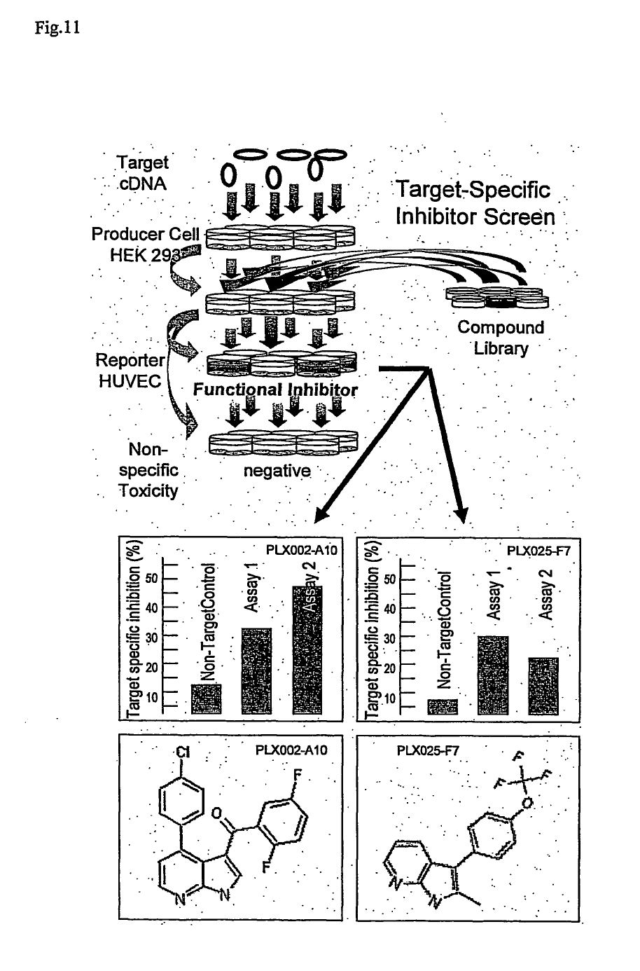

The screen for low molecular compounds inhibiting TBK-1 induced active was performed with the screening assay described in example 1 supplemented by addition of individual compounds to the transfected producer cells HEK293 (see FIG. 11). HEK293 cells were transfected with an expression plasmid for TBK-1, Toll-interleukin 1 receptor domain (TIR)-containing adaptor molecule-1 (TICAM-1, an upstream activator of TBK-1) or VEGF as control. 4 hours after transfection compounds were added at a final concentration of 25 μM. 48 hours after transfection supernatants were transferred to HUVEC cells. Proliferation of HUVEC cells was measured after 5 days using the Alamar Blue Assay as read out.

Examples of the results of these analyses are shown in FIG. 11: inhibition of TBK-1 (assay 1) and TICAM-1 (assay 2) induced proliferative activity of supernatants on HUVEC cells by indicated compounds. VEGF served as non-target control.

To further characterize the inhibitory potential of the compounds, dose response experiments were performed in which various concentrations of compound were applied to the transfected cells.

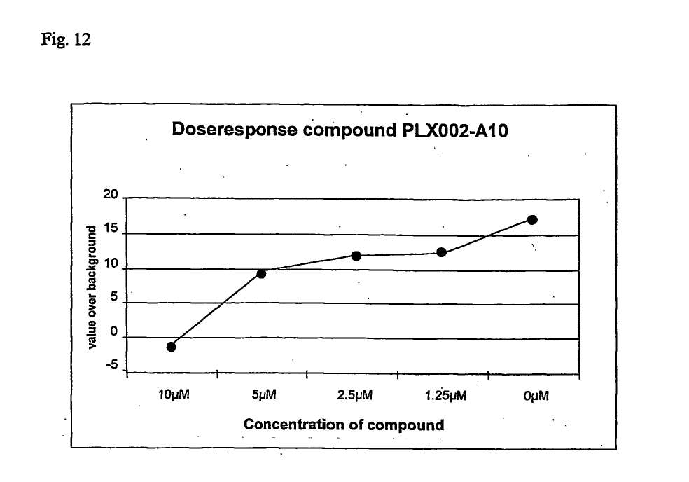

FIG. 12 shows the dose dependent inhibition of TBK-1 with the compound PLX002-A10 as an example. As expected for a compound which acts specifically a dose dependent reduction of the signals was observed.

To further proof the specific inhibitory action of compounds, some selected compounds from the screen were subjected to biochemical analyses in which inhibition of the activity of recombinant TBK-1 protein and inhibition of IKK B (a substrate for TBK-1) phosphorylation by TBK-1 was determined.

For these experiments, recombinant His-tagged TBK-1 protein was produced in SF-9 baculo cells and purified via Ni-affinity chromatography. For determination of inhibition of enzymatic activity of TBK-1 we used fluorescence polarization to measure the displacement of labelled adenosinetriphosphate (ATP) by individual compounds and therefore binding of the compound to the catalytic domain of TBK-1.

For detection of IKKβ phosphorylation in Western Blot experiments and inhibition of phosphorylation we used an anti-phospho-IKKα/β antibody (Cell Signaling).

In these experiments a compound could be identified which binds to the catalytic domain of TBK-1 recombinant protein and inhibits IKKβ phosphorylation in TBK-1 transfected cells. This compound also specifically inhibits TBK-1 induced activity in transfected HEK293 cells in a dose dependent manner.

1. A method of treating a patient having an ischemic disease, a dental disease, smoker's leg, a diabetic ulcer, or stimulation of wound healing in said patient in need thereof, said method comprising administering a nucleic acid encoding TBK-1 or a functional active derivative thereof.

2. The method of claim 1, wherein the nucleic acid induces the production of VEGF.

3. The method according to any of claims 1 or 2, wherein the nucleic acid induces the formation of vascular vessels.

4. A method for the diagnosis of ischemic disease, a dental disease, smoker's leg, a diabetic ulcer, a wound healing disorder, cancer, hyperplasia, tumor progression, rheumatoid arthritis, psoriasis, artherosclerosis, retinopathy, osteoarthritis, endometriosis or chronic inflammation in a patient, said method comprising detecting the level of
a) TBK-1,
b) a functional active derivative thereof, and/or
c) a nucleic acid encoding TBK-1,
said method comprising the use of a means for the detection of the molecules of sections a), b), or c).

5. A method for inhibiting or reducing angiogenesis in the treatment of cancer, hyperplasia, rheumatoid arthritis, psoriasis, artherosclerosis, retinopathy, osteoarthritis, endometriosis or chronic inflammation in a patient in need thereof, said method comprising administering a TBK-1 inhibitor.

6. The method of claim 5, wherein the inhibitor inhibits the production of VEGF.

7. The method of any of claims 5 or 6, wherein the inhibitor inhibits the formation of vascular vessels.

8. The method of any of claims 5 or 7, wherein the inhibitor is selected from the group consisting of antisense oligonucleotides, antisense RNA, siRNA, aptamers and Low molecular weight molecules (LMWs).

9. The method of claim 8, wherein the LMWs bind to the ATP-binding site of the kinase domain of TBK-1.

10. The method of any of claims 4 to 9, wherein the disease is cancer, preferably selected from the group consisting of brain cancer, pancreas carcinoma, stomach cancer, colon carcinoma, skin cancer, especially melanoma, bone cancer, kidney carcinoma, liver cancer, lung carcinoma, ovary cancer, mamma carcinoma, uterus carcinoma, prostate cancer and testis carcinoma.

11. A method for the identification of an anti-cancer drug, wherein
a) a potential TBK-1 interactor is brought into contact with TBK-1 or a functional derivative thereof, and
b) binding of the potential interactor to TBK-1 or the functional derivative thereof is determined, and
c) the anti-angiogenic capacity of the potential interactor is determined.

12. The method of claim 11, wherein the anti-angiogenic capacity is determined by measuring the inhibition of VEGF production.

13. The method of any of claims 11 or 12, wherein the potential interactor is provided in the form of a chemical compound library.

14. The method of claim 13, wherein the chemical compound library consists of a group of molecules or substances that bind to the ATP binding site of the kinase domain of TBK-1.

15. The method of any of claims 11 or 14, wherein the method is carried out on an array.
